(19) United States
(12) Patent Application Publication
(10) Pub. No.: US 2008/0004518 A1
(43) Pub. Date: Jan. 3, 2008

(54) MAGNETIC RESONANCE SEQUENCE FOR QUANTITATIVE T1 MAPPING DURING FREE BREATHING

(75) Inventors: Christian STEHNING, Hamburg (DE); Tobias R. SCHAEFFTER, Blackheath (GB)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/769,946

(22) Filed: Jun. 28, 2007

Related U.S. Application Data
(60) Provisional application No. 60/806,109, filed on Jun. 29, 2006.

(51) Int. Cl. A61B 5/055

(52) U.S. Cl. 600/410

(57) ABSTRACT

An apparatus includes a magnetic resonance scanner configured to apply a navigation pulse exciting a navigation region, and a saturation or inversion pulse saturating or inverting a region of interest but not saturating or inverting a portion or all of the navigation region, and to read navigation magnetic resonance data excited by the navigation pulse and informational magnetic resonance data in the saturated or inverted region of interest. A processor is configured to process the informational magnetic resonance data based at least in part on the navigation magnetic resonance data. The apparatus is suitable for performing an imaging method including: saturating or inverting an imaging region while leaving a navigation region unsaturated or non-inverted; generating navigation data from the navigation region; generating saturation or inversion recovery data from the imaging region; and creating a T1 map from the saturation or inversion recovery data.

### CROSS REFERENCE TO RELATED APPLICATIONS

This application claims the benefit of U.S. provisional application Ser. No. 60/806,109 filed Jun. 29, 2006, which is incorporated herein by reference.

### BACKGROUND

The present application relates to the magnetic resonance arts. It is described with particular reference to T1 mapping of the myocardium (that is, the muscular tissue of the heart). However, the following is amenable to other applications such as T1 mapping in general, magnetic resonance imaging or spectroscopy employing saturation or inversion pulses, and so forth.

T1 mapping involves measuring or estimating the T1 relaxation time of excited magnetic resonance as a function of spatial position. A T1 map provides an image indicative of spatial variation of T1 relaxation time. Since the T1 relaxation time is different for different types of tissues and other substances, the T1 map can provide suitable image contrast for various clinical diagnostic applications.

In one application, T1 mapping advantageously provides useful information about the myocardial tissue including diagnosis of plaque buildup and diagnosis of other myocardial tissue abnormalities. Higgins et al., “T1 measurement using a short acquisition period for quantitative cardiac applications”, Med. Phys. vol. 32(6), pp. 1738-46 (2005) discloses an approach for applying T1 mapping to myocardial tissue. In this approach, a saturation pulse is applied, and magnetic resonance images are acquired at several delay times (T_{S}) respective to the saturation pulse to measure the magnetic resonance signal recovery rate. The characteristic T1 value is then mapped based on the magnetic resonance signal recovery rate mapped by the several acquired images. To account for cardiac motion, the cardiac cycle is monitored by an electrocardiograph (ECG), and one magnetic resonance image is acquired, at the same cardiac phase, per cardiac cycle. To acquire blood flow information, a bolus of an intravenous contrast agent with a distinct T1 value, such as Gd-DTPA, may be administered.

The approach of Higgins has certain disadvantages. Although cardiac motion is taken into account using the ECG, respiratory motion is not. Rather, the approach of Higgins requires that the patient hold his or her breath during the imaging. Since only a single magnetic resonance image is typically acquired per heartbeat (approximately one image per second), and several magnetic resonance images are typically desirable to accurately spatially map the T1 value, the requirement of a breath hold during imaging substantially limits the acquisition time. This limitation is especially problematic in the case of elderly or ill patients who may have shortened breath hold times. Higgins suggests extending the acquisition time by performing imaging over multiple breath holds; however, this introduces substantial likelihood of patient movement between breath holds and consequent motion artifacts in the T1 mappings.

Börnert et al., U.S. Pat. No. 5,977,769 discloses a respiratory monitoring technique in which a two-dimensional radio frequency pulse excites the nuclear magnetization along a line in temporal cooperation with two oscillating magnetic gradient fields. The line of excitation is chosen to substantially transversely intersect the diaphragm of the patient. The excited line of magnetic resonance is read out using a suitable read gradient, and is reconstructed to produce a linear projection from which the movement of the diaphragm can be deduced so as to monitor respiration. However, the respiratory monitoring of Börnert is not compatible with the T1 mapping approach of Higgins, because the saturation pulse of Higgins would interfere with the excitation of a line of magnetic resonance as in Börnert.

The following provides improvements which overcome the above-referenced problems and others.

### SUMMARY

In accordance with one aspect, a magnetic resonance method is disclosed. An imaging region is saturated or inverted, while leaving a navigation region unsaturated or non-inverted. Navigation data are generated from the navigation region. Saturation recovery or inversion recovery data are generated from the imaging region. A T1 map is created from the saturation recovery or inversion recovery data.

In accordance with another aspect, a magnetic resonance apparatus is disclosed. A magnetic resonance scanner is configured to (i) apply a navigation pulse exciting a navigation region, and a saturation or inversion pulse saturating or inverting a region of interest but not saturating or inverting a portion or all of the navigation region, and to (ii) read navigation magnetic resonance data excited by the navigation pulse and informational magnetic resonance data in the region of interest saturated or inverted by the saturation pulse. A processor is configured to process the informational magnetic resonance data based at least in part on the navigation magnetic resonance data.

In accordance with another aspect, a magnetic resonance imaging apparatus is disclosed. Means are provided for monitoring respiratory phase. Means are provided for monitoring cardiac phase. Means are provided for performing a saturation recovery or inversion recovery sequence including applying a saturation or inversion pulse and reading saturation recovery or inversion recovery data. The performing means communicates with the cardiac phase monitoring means to read the saturation recovery or inversion recovery data at about a selected cardiac phase. The performing means varies a temporal offset between the applying of the saturation or inversion pulse and the reading during successive heartbeats to sample different portions of a saturation or inversion recovery curve. Means are provided for generating a T1 map from the saturation recovery or inversion recovery data. Means are provided for respiratory gating communicating with the respiratory phase monitoring means to ensure that the T1 map is generated from saturation recovery or inversion recovery data read while the respiratory phase is in a selected respiratory phase window.

One advantage resides in providing T1 mapping of the myocardium or other tissue affected by respiration during free breathing.

Another advantage resides in simultaneous T1 mapping and magnetic resonance-based respiratory monitoring.

Another advantage resides in simultaneous imaging employing saturation pulses and magnetic resonance-based respiratory monitoring.

Another advantage resides in reduced artifacts due to cyclic motion such as respiration during imaging, T1 mapping, and the like.

Still further advantages of the present invention will be appreciated to those of ordinary skill in the art upon reading and understand the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically shows a magnetic resonance system including respiratory gating and cardiac gating.

FIG. 2 diagrammatically shows timing of the saturation pulse, navigator sub-sequence, and acquisition interval of a T1 mapping sequence in the context of a concurrent electrocardiographic signal, respiratory signal, and an inversion or saturation recovery curve.

### DETAILED DESCRIPTION

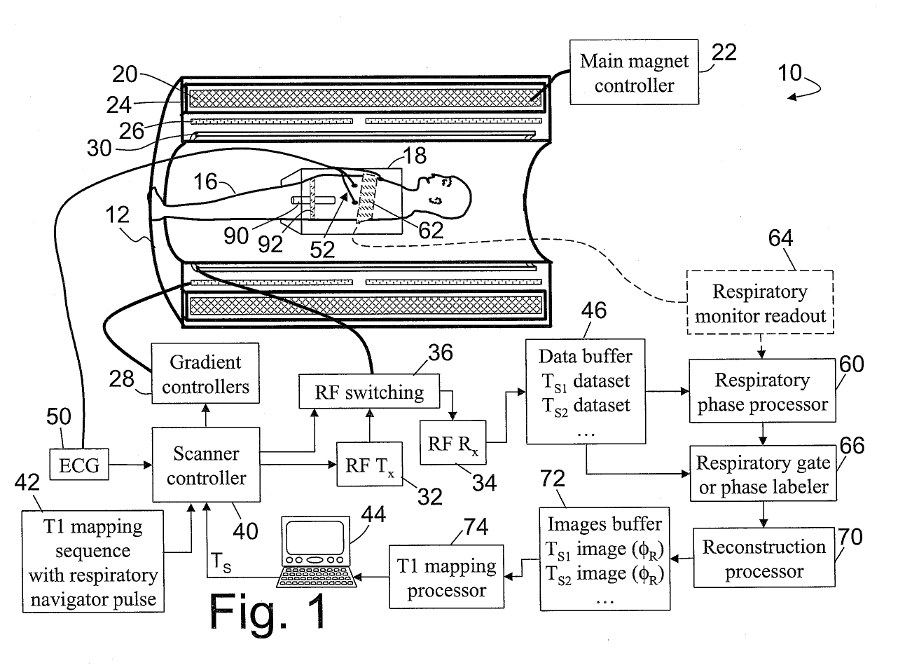

With reference to FIG. 1, a magnetic resonance scanner 10 includes a scanner housing 12 in which a patient 16 or other subject is at least partially disposed with a heart or other organ or anatomical region to be studied positioned in a scanning region 18 of the scanner 10. Although described with reference to a bore-type scanner, it is to be appreciated that the scanner could also be an open-magnet scanner or other type of magnetic resonance scanner. A main magnet 20 disposed in the scanner housing 12 is controlled by a main magnet controller 22 to generate a static (B₀) magnetic field in at least the scanning region 18. Typically, the main magnet 20 is a persistent superconducting magnet surrounded by cryoshrouding 24, although a resistive magnet can also be used. In some embodiments, the main magnet 20 generates a main magnetic field of between about 0.23 Tesla and about 7 Tesla; however, main magnetic fields of strengths above or below this typical range are also contemplated. A gradient system including magnetic field gradient coils 26 arranged in or on the housing 12 and corresponding gradient controllers 28 superimpose selected magnetic field gradients on the main magnetic field in at least the scanning region 18. Typically, the magnetic field gradient coils 26 include coils for producing three orthogonal magnetic field gradients, such as x-, y-, and z-gradients.

A generally cylindrical whole-body coil 30 is mounted substantially coaxially with the bore of the magnetic resonance scanner 10. The whole-body coil 30 may be, for example, a quadrature birdcage coil, transverse electromagnetic (TEM) coil, or so forth. Additionally or alternatively, one or more local radio frequency coils such as a surface coil or plurality of surface coils, a SENSE coil array, a torso coil, or so forth (not shown) can be employed. In the embodiment of FIG. 1, the whole-body coil 30 performs both transmit and receive functions. That is, the whole-body coil 30 is energized at a magnetic resonance frequency by one or more radio frequency transmitters 32 to excite magnetic resonance in the subject 16, and the whole-body coil 30 is also used in conjunction with one or more radio frequency receivers 34 to receive magnetic resonance signals emanating from the subject 16 responsive to such excitation. Suitable radio frequency switching circuitry 36 are provided to enable the whole-body coil 30 to perform both transmit and receive functions.

While shown as a separate unit, in some embodiments the radio frequency switching circuitry or portions thereof may be integrated into the whole-body coil, the radio frequency transmitter, or the radio frequency receiver. In other contemplated embodiments, the whole-body coil 30 performs the transmit function, while one or more local radio frequency coils receives the generated magnetic resonance signals. In other contemplated embodiments, the whole-body coil 30 is omitted and one or more local radio frequency coils perform both transmit and receive functions. It is still further contemplated to use the whole-body coil 30 as a receive coil while magnetic resonance is excited using one or more local radio frequency coils.

The magnetic resonance scanner 10 operates under the control of a scanner controller 40 to perform a selected magnetic resonance sequence 42, such as the example T1 mapping sequence with respiratory navigator pulse which is described herein. A user interface 44 enables a radiologist or other user to select the sequence 42 or another magnetic resonance sequence, and also enables the user to set or modify parameters of the sequence such as a T_{S} temporal offset parameter of the example T1 mapping sequence with respiratory navigator pulse 42. The scanner 10 operates under the control of the scanner controller 40 in accordance with the selected sequence 42 to excite magnetic resonance and generate magnetic resonance data that are stored in a data memory or buffer 46. The sequence is re-executed to generate multiple sets of data, such as the illustrated T_{S}, dataset, T_{S2} dataset, . . . shown in the data buffer 46 corresponding to re-executing the selected sequence 42 with different values for the temporal offset parameter T_{S}. Optionally, an electrocardiograph 50 with leads 52, or additional or other auxiliary equipment, monitors the patient 16 during the magnetic resonance data acquisition. For example, the ECG 50 can provide cardiac gating information to ensure that data is acquired at about a selected cardiac phase such as at about the diastolic phase or about the systolic phase. In some embodiments, the generating of saturation recovery or inversion recovery data is cardiac gated using the ECG 50 such that data are acquired in multiple cardiac phases, and multiple saturation recovery or inversion recovery data sets are derived, in which each data set is assigned to a selected cardiac phase.

The example sequence 42 includes navigator pulses which produce navigator magnetic resonance data that is suitably analyzed by a respiratory phase monitor 60 to determine a respiratory phase of the patient 16 during execution of the sequence 42. Additionally or alternatively, a designated respiratory monitor, such as illustrated respiratory bellows 62 and associated respiratory monitor readout 64, provides input data from which the respiratory phase monitor 60 determines the respiratory phase of the patient 16. (The optional nature of the dedicated respiratory monitor 62, 64 is indicated in FIG. 1 by depicting these components using dashed lines). The respiratory phase information is by a respiratory gate or phase labeler 66 to select magnetic resonance data for further use, or to label the acquired magnetic resonance data with respiratory phase to enable further processing to take into account artifacts that may be attributable to respiratory motion.

The following description refers to generating T1 maps from saturation recovery data. It is also contemplated to acquire inversion recovery data, and to generate the T1 maps from the inversion recovery data. A reconstruction processor 70 reconstructs the acquired magnetic resonance data, or portions thereof selected by the respiratory gate 66, into a reconstructed image. In the illustrated embodiment, each re-execution of the T1 mapping sequence 42 generates a separate informational magnetic resonance dataset, such as the example T_{S1} and T_{S2} saturation recovery datasets acquired with the temporal offset parameter T_{S} having values T_{S1} and T_{S2}, respectively, for successive executions of the sequence 42. These datasets are each reconstructed into a reconstructed image by the reconstruction processor 70, so as to for example generate reconstructed T_{S1} and T_{S2} images, and so forth, which are suitably stored in an images memory or buffer 72. If the respiratory gate or phase labeler 66 labels the data with respiratory phase, then the resulting reconstructed images are suitably labeled by the respiratory phase, such as the illustrated respiratory phase labels φ_{R} shown associated with the images in the buffer 72. This phase information is optionally used to perform retrospective respiratory gating at the post-image reconstruction level, for example by selectively storing only those images with assigned respiratory phase in a desired range in the images buffer 72. Alternatively, the respiratory gate or phase labeler 66 may perform a correction of respiration-induced translational motion or deformation. For example, saturation recovery or inversion recovery data may be included in the processing after correction of respiratory motion induced displacement or deformation, or image artifacts resulting from respiratory motion occurring during data acquisition. Processing in addition to or instead of image reconstruction can also be performed on the informational magnetic resonance data. For example, the reconstructed images acquired using the T1 mapping sequence 42 are suitably processed by a T1 mapping processor 74 to generate a T1 map of the imaged region. In some embodiments, the T1 map is derived from the saturation recovery or inversion recovery data using a technique that does not involve reconstruction of intermediate images. The T1 map is suitably displayed on the user interface 44 or on another display device, or may be printed, communicated over the Internet or a local area network, stored on a non-volatile storage medium, or otherwise used. In the example configuration illustrated in FIG. 1, the user interface 44 performs both scanner control interfacing and data display and analysis tasks; however, it is also contemplated to have separate scanner control interfacing and data display and/or analysis computers or systems.

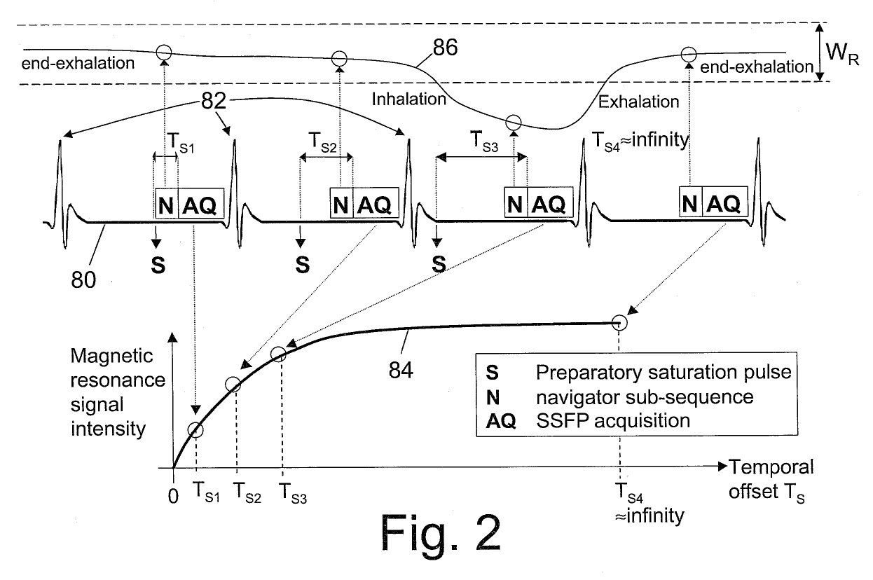

With continuing reference to FIG. 1 and with further reference to FIG. 2, the example T1 mapping sequence with respiratory navigator pulse 42 is described in greater detail. The sequence is performed in conjunction with cardiac gating based on an ECG signal 80 acquired by the ECG 50. In a suitable approach, prominent R-wave peaks 82 of the ECG signal 80 are used as temporal markers indicative of repetitions of the cardiac cycle. In other embodiments, other features of the ECG signal 80 may be used to demark the cardiac cycle, such as T-wave or so forth. Moreover, other devices besides the illustrated example ECG 50 can provide cardiac cycling information, such as an echocardiograph, magnetic resonance navigator pulses, or so forth. Informational magnetic resonance data, such as magnetic resonance imaging data, is acquired during an acquisition interval AQ indicated in FIG. 2. In the illustrated embodiment, the acquisition interval AQ is substantially offset from the QRS complex demarked by the R-wave peak 82. The QRS complex corresponds approximately to activation of the ventricle, and thus corresponds to a region in which the cardiac muscle is in motion. The illustrated acquisition interval AQ is in the late diastole period in which the cardiac muscle is substantially relaxed and quiescent. The acquisition interval AQ is also advantageously located at the same temporal offset from the R-wave peak 82 in each heartbeat so that the heart is in substantially the same cardiac phase during each acquisition interval AQ.

The illustrated example T1 mapping sequence 42 is a saturation recovery-type steady-state free precession (SSFP) sequence in which the spins are saturated by a saturation pulse S and the information magnetic resonance data acquired using an SSFP acquisition readout during the acquisition interval AQ arranged at a time interval TS offset from the saturation pulse S. The informational magnetic resonance data acquired during the acquisition interval AQ indicates the extent of recovery of the magnetic resonance signal over the time interval T_{S}. A magnetic resonance signal recovery curve 84 plotted in FIG. 2 is related to the T1 value of the excited tissue or other material. To sample different points along the recovery curve 84, the temporal offset T_{S} between the magnetization preparation pulse S and the data acquisition interval AQ is varied from cardiac cycle to cardiac cycle. In FIG. 2, for example, the first, second, and third cardiac cycles employ successively longer temporal offset intervals T_{S1}, T_{S2}, and T_{S3}. In the fourth cardiac cycle, no saturation pulse is applied and so the temporal offset interval T_{S4} is in effect infinite to sample the boundary value of the relaxation curve 84.

The SSFP acquisition is advantageously an imaging acquisition that spatially encodes the acquired informational magnetic resonance data such that the reconstruction processor 70 produces an image indicative of the spatial distribution of magnetic resonance signal intensity corresponding to each acquisition AQ. Accordingly, by processing these reconstructed images on a pixel-by-pixel or voxel-by-voxel basis using the T1 mapping processor 74, a spatial map of the T1 value is obtained.

To account for respiratory motion, each execution of the T1 mapping sequence 42 includes a navigator sub-sequence N to acquire respiratory phase information. The navigator sub-sequence N is advantageously disposed close in time to the acquisition interval AQ, so that the respiratory phase determined by the navigator sub-sequence N is close to the respiratory phase during the acquisition interval AQ. This approximation is typically accurate since the respiratory cycle length (typically one breath every 10-15 seconds) is substantially longer than the cardiac cycle length (typically one heartbeat every second or so). Accordingly, the respiratory phase determined by the navigator sub-sequence N is assigned to the acquisition interval AQ within the same heartbeat. As shown in FIG. 2, each application of the navigator sub-sequence N produces a sample of a respiratory-related signal 86 indicative of a respiratory cycle. In some embodiments, the respiratory gate or phase labeler 66 performs gating by performing processing on the informational magnetic resonance data acquired during that heartbeat only if the respiratory phase indicated by the navigator sub-sequence N lies within a selected respiratory phase window W_{R}. Thus, in the example of FIG. 2, the informational magnetic resonance data acquired in conjunction with the T_{S3} offset would be discarded since the respiratory phase for that data lies outside of the illustrated respiratory phase window W_{R}. In other embodiments, the respiratory gate or phase labeler 66 performs gating by labeling the informational magnetic resonance data acquired during a heartbeat and stored in the images buffer 72 with the respiratory phase (φ_{R}) indicated by the corresponding navigator sub-sequence N. The respiratory phase label (φ_{R}) can then be used to sort, select, or otherwise control or limit further processing of the reconstructed images, for example by the T1 mapping processor 74.

The navigator sub-sequence N and corresponding processing performed by the respiratory phase processor 60 in some embodiments is based on the navigation method of Börnert et al., U.S. Pat. No. 5,977,769. The navigator sub-sequence N includes a radio frequency navigation pulse applied in conjunction with spatially selective magnetic field gradients to excite magnetic resonance along a one-dimensional navigation region, such as the illustrated extended length, small cross-sectional area cylinder navigation region 90 shown in FIG. 1. The cross-section can be as small as one or a few voxels. The one-dimensional navigation region 90 is arranged substantially transverse to a diaphragm 92 of the patient 16, and the length of the navigation region 90 should be sufficient to span the range of movement of a diaphragm 92 during respiration. The navigator sub-sequence N further includes a readout that acquires navigation magnetic resonance data excited in the one-dimensional navigation region 90 by the navigator pulse. The navigator readout is performed in conjunction with a suitable frequency-encoding readout magnetic field gradient, and the resulting navigation magnetic resonance data is reconstructed by the respiratory phase processor to produce a linear projection from which the movement of the diaphragm 92 can be deduced so as to monitor respiration.

In some embodiments, the navigation pulse is applied in conjunction with a slice-selective magnetic field gradient so that the navigation region is a two-dimensional slice or slab. It is to be appreciated that the one- or two-dimensional navigation region may have some breadth. For example, a one-dimensional navigation region may be an elongated cylinder having a small cross-section, while a two-dimensional navigation region may be a thin slab having some finite thickness. Because the diaphragm 92 presses against the air-filled lungs (not illustrated), a readout of magnetic resonance signal along the navigation region 90 shows an abrupt signal change at the interface between the diaphragm 92 and the lungs, providing a spatially localized signal indicative of respiratory phase.

With continuing reference to FIGS. 1 and 2, the preparatory saturation pulse S of the T1 mapping sequence with respiratory navigator pulse 42 is modified to ensure that the saturation pulse S does not interfere with the navigator sub-sequence N. If the saturation pulse was to saturate the navigation region 90 (or was to saturate the operative portion of the navigation region 90 that intersects with the diaphragm 92) then there would be insufficient magnetization for the navigator sub-sequence N to function as intended. Rather, the saturation pulse S is spatially tailored to saturate the region of interest, such as the heart or other organ or anatomical feature of interest, while excluding the navigation region 90 (or excluding at least the operative portion of the navigation region 90 that intersects with the diaphragm 92).

In one suitable approach, the saturation pulse S includes two components: a first excitation pulse component having a first flip angle, and a second excitation pulse component having a second flip angle equal in magnitude and opposite in polarity to the first flip angle. For example, the first and second excitation pulse components may have flip angles of +90° and −90°, respectively, or the first and second excitation pulse components may have flip angles of −90° and +90°, respectively. Flip angle magnitudes of other than 90° are also contemplated. For inversion recovery, the first and second excitation pulse components typically have equal magnitude and arbitrary polarity. For example, the first and second excitation pulse components can both be +180° pulses, or can be both −180° pulses, for inversion recovery. The first excitation pulse component excites a first region that includes at least the operative portion of the navigation region 90 that intersects the diaphragm 92. In some embodiments, the first region is the same spatial region as the navigation region 90. In other embodiments, the first region may be a slab oriented parallel with and encompassing the diaphragm 92, with sufficient slab thickness to encompass the diaphragm 92 throughout the respiratory cycle. Other geometries are possible for the first region. The second excitation pulse component excites a second region that encompasses the first region and the region of interest, such as the heart or other organ or anatomical feature of interest. In some embodiments, the second excitation pulse is a spatially non-selective excitation pulse.

It is to be appreciated that the first and second excitation pulse components of the saturation pulse S may be applied in either order: that is, the first excitation pulse component may be applied first, followed by the second excitation pulse component, or the second excitation pulse component may be applied first, followed by the first excitation pulse component.

The effect of the described two-component saturation pulse S is as follows. In the region of interest, only the second excitation pulse component is applied. Accordingly, the region of interest “sees” the desired preparation pulse (for example, a +90° saturation pulse, or a −90° saturation pulse). Because the region of interest is saturated by the second excitation pulse component, the temporal offset T_{S} between the magnetization preparation pulse S and the data acquisition interval AQ is defined respective to the second excitation pulse component. On the other hand, in the first region which includes at least the operative portion of the navigation region 90 that intersects the diaphragm 92, both the first and second excitation pulse components are applied. Accordingly, the first region “sees” both the first and second excitation pulse components. Because the first and second excitation pulse components have flip angles of equal magnitude but opposite polarity, these excitation pulse components cancel in the first region. Accordingly, the first region is not saturated, and so the navigation sub-sequence N can operate as intended to provide respiratory phase information. The time difference between application of the first and second excitation pulse components should be short, to provide effective cancellation in the first region.

Other arrangements are contemplated for producing the spatially selective saturation pulse that saturates a region of interest but does not saturate an operative portion or all of the navigation region. For example, if the region of interest is a thin slice or slab, it is contemplated to use a single saturation pulse along with a slice-selective magnetic field gradient that positions the saturation pulse on the thin slice or slab region of interest. However, in some approaches the saturation pulse must saturate a relatively large region. For example, if a steady state imaging technique is used to image a volume, then the volume to be imaged is maintained in steady state and should be saturated by the saturation pulse. If this volume is large or non-planar, it may not be possible to suitably confine the saturation pulse to the volume of interest without impinging upon the operative portion of the navigation region using a concurrent slice-selective magnetic field gradient. The two-component saturation pulse S is suitably used in such cases to saturate the substantial volume while not saturating at least the operative portion of the navigation region.

The T1 mapping sequence with respiratory navigator pulse 42 is an illustrative example. More generally, informational magnetic resonance data is acquired using substantially any type of imaging, mapping, or spectroscopy sequence that employs a preparatory pulse. The informational magnetic resonance data may, for example, be imaging data acquired using saturation recovery, steady state free precession imaging, a spoiled gradient echo sequence, or so forth. The navigation sub-sequence includes substantially any spatially limited navigation pulse that excites a navigation region followed by a suitable readout to generate navigation magnetic resonance data. The navigation sub-sequence can be configured to derive various types of navigation information, such as the described respiratory phase information, or cardiac phase information, or spatial registration information for registering images acquired at different times or by different imaging modalities, or information on the progress of an injected magnetic contrast agent bolus, or so forth. The preparatory pulse is spatially selective. This is accomplished in some embodiments by dividing the preparatory pulse into first and second excitation pulse components. The first excitation pulse component has a first flip angle and is spatially selective to excite a first region that includes an operative portion of the navigation region (such as the intersection of the navigation region 90 with the diaphragm 92 in the illustrated example). The second excitation pulse component has a second flip angle equal in magnitude and opposite in polarity to the first flip angle, and excites a second region including at least the first region and a region of interest. The second excitation pulse component may be spatially non-selective.

In other contemplated embodiments, the respiratory phase is acquired using a non-magnetic resonance technique such as the illustrated respiratory bellows 62 and associated respiratory monitor readout 64, in which case the preparatory pulse can be spatially non-selective. Optionally, a bolus of an intravenous contrast agent with a distinct T1 value or other magnetic characteristic, such as Gd-DTPA, may be administered prior to or during acquisition of the informational magnetic resonance data so as to provide dynamic blood flow information or other information. Moreover, the informational magnetic resonance data can be configured to provide magnetic resonance spectroscopy information in conjunction with or instead of imaging or spatial mapping information.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

Having thus described the preferred embodiments, the invention is now claimed to be:

1. A magnetic resonance method comprising:
saturating or inverting an imaging region while leaving a navigation region unsaturated or non-inverted;
generating navigation data from the navigation region;
generating saturation recovery or inversion recovery data from the imaging region; and
creating a T1 map from the saturation recovery or inversion recovery data.

2. The magnetic resonance method as set forth in claim 1 wherein the saturating or inverting comprising:
applying a first excitation pulse component to excite a first region that includes at least an operative portion of the navigation region; and
applying a second excitation pulse component to excite at least the imaging region and the first region, the first and second excitation pulse components substantially canceling in the first region.

3. The magnetic resonance method as set forth in claim 2, wherein the first and second excitation pulse components have one of (i) respective first and second flip angles that are equal in magnitude and opposite in polarity or (ii) equal magnitude.

4. The magnetic resonance method as set forth in claim 2, wherein the first region includes at least a portion of the navigation region that intersects a diaphragm, the magnetic resonance method further including:
assigning respiratory phase values to the inversion recovery data based on substantially concurrently generated navigation data.

5. The magnetic resonance method as set forth in claim 4, wherein the navigation region is a one- or two-dimensional navigation region arranged substantially transverse to the diaphragm.

6. The magnetic resonance method as set forth in claim 5, wherein the first region coincides with the navigation region.

7. The magnetic resonance method as set forth in claim 5, wherein the first region encompasses with the diaphragm or other structure that is subject to respiratory motion.

8. The magnetic resonance method as set forth in claim 4, wherein the creating of the T1 map from the saturation recovery or inversion recovery data includes one of:
excluding saturation recovery or inversion recovery data having assigned respiratory phase values outside of a selected respiratory phase window; or
including saturation recovery or inversion recovery data after correction of respiratory motion induced displacement or deformation, or image artifacts resulting from respiratory motion occurring during data acquisition.

9. The magnetic resonance imaging method as set forth in claim 8, wherein the saturating or inverting and the generating saturation recovery or inversion recovery data are repeated with different time intervals between the saturating or inverting and the generating to acquire saturation recovery or inversion recovery data with different magnetic resonance recovery times, and the creating of the T1 map includes:
deriving a T1 map from the saturation recovery or inversion recovery data with different magnetic resonance recovery times.

10. The magnetic resonance imaging method as set forth in claim 9, further including at least one of:
cardiac gating the generating of saturation recovery or inversion recovery data such that the saturation recovery or inversion recovery data is read over a plurality of cardiac cycles at about the same cardiac phase during each cardiac cycle; and
Cardiac gating the generating of saturation recovery or inversion recovery data such that data are acquired in multiple cardiac phases, and multiple saturation recovery or inversion recovery data sets are derived, in which each data set is assigned to a selected cardiac phase.

11. The magnetic resonance method as set forth in claim 2, wherein the first and second excitation pulses satisfy one of the following criteria:
(i) the first flip angle is one of 90° and −90° and the second flip angle is the other of 90° and −90°; or
(ii) the first flip angle and the second flip angle are both 180°, irrespective of polarity.

12. A magnetic resonance apparatus comprising:
a magnetic resonance scanner configured to (i) apply a navigation pulse exciting a navigation region, and a saturation or inversion pulse saturating or inverting a region of interest but not saturating or inverting a portion or all of the navigation region, and to (ii) read navigation magnetic resonance data excited by the navigation pulse and informational magnetic resonance data in the region of interest saturated by the saturation or inversion pulse; and
a processor configured to process the informational magnetic resonance data based at least in part on the navigation magnetic resonance data.

13. The magnetic resonance apparatus as set forth in claim 12, wherein the saturation or inversion pulse includes:
a first excitation pulse component exciting the portion or all of the navigation region and excluding the region of interest; and
a second excitation pulse component exciting at least the portion or all of the navigation region and the region of interest, the first and second excitation pulse components substantially canceling in a region of overlap.

14. The magnetic resonance method as set forth in claim 13, wherein the first excitation pulse component has a flip angle of one of 90° or −90° and the second excitation pulse component has a flip angle of the other of 90° or −90°.

15. The magnetic resonance apparatus as set forth in claim 12, wherein the processor includes:
a respiratory gate operative to identify informational magnetic resonance data acquired in a selected respiratory phase window as indicated by substantially concurrently acquired navigation magnetic resonance data.

16. The magnetic resonance apparatus as set forth in claim 15, wherein the informational magnetic resonance data are acquired at different magnetic resonance recovery times respective to the saturation or inversion pulse, and the processor further includes:
a T1 mapping processor that generates a T1 map based on the informational magnetic resonance data acquired at different magnetic resonance recovery times respective to the saturation or inversion pulse.

17. The magnetic resonance apparatus as set forth in claim 16, further including:
a cardiac monitor configured to gate or trigger the magnetic resonance scanner such that the informational magnetic resonance data is read at about a selected cardiac phase.

18. A magnetic resonance imaging apparatus comprising:
means for monitoring respiratory phase;
means for monitoring cardiac phase;
means for performing a saturation recovery or inversion recovery sequence including applying a saturation or inversion pulse and reading saturation recovery or inversion recovery data, the performing means communicating with the cardiac phase monitoring means to read the saturation recovery or inversion recovery data at about a selected cardiac phase, the performing means varying a temporal offset between the applying of the saturation or inversion pulse and the reading during successive heartbeats to sample different portions of a saturation recovery or inversion recovery curve;
means for generating a T1 map from the saturation recovery or inversion recovery data; and
means for respiratory gating communicating with the respiratory phase monitoring means to ensure that the T1 map is generated from saturation recovery or inversion recovery data read while the respiratory phase is in a selected respiratory phase window.

19. The magnetic resonance imaging apparatus as set forth in claim 18, wherein the respiratory phase monitoring means includes:
means for applying a navigation sub-sequence including a navigation pulse that excites magnetic resonance in a navigation region and a readout that reads navigation magnetic resonance data excited by the navigation pulse,
wherein the saturation or inversion pulse of the saturation recovery or inversion recovery sequence is a spatially selective saturation or inversion pulse that does not saturate or invert at least an operative portion of the navigation region.

20. The magnetic resonance imaging apparatus as set forth in claim 19, wherein the saturation or inversion pulse of the saturation or inversion recovery sequence is divided into first and second excitation pulse components in which the second excitation pulse component saturates or inverts a region from which the saturation or inversion recovery imaging data is read and the first excitation pulse component substantially cancels the second excitation pulse component at least in the operative portion of the navigation region.

21. The magnetic resonance imaging apparatus as set forth in claim 18, wherein the region from which the inversion recovery imaging data is read includes tissue that is subject to cardiac and respiratory motion, such as myocardial tissue.
